(19) United States
(12) Patent Application Publication
(10) Pub. No.: US 2026/0007500 A1
(43) Pub. Date: Jan. 8, 2026

(54) MAXILLA HOLDER AND INSTALLATION THEREOF

(71) Applicant: Liang-Cheng CHEN, Taichung City (TW)

(72) Inventor: Liang-Cheng CHEN, Taichung City (TW)

(21) Appl. No.: 19/249,301

(22) Filed: Jun. 25, 2025

Related U.S. Application Data
(62) Division of application No. 18/813,861, filed on Aug. 23, 2024, now Pat. No. 12,357,434.

(30) Foreign Application Priority Data
Aug. 25, 2023 (TW) 112132179

(51) Int. Cl. A61C 11/00; A61B 17/66; A61B 17/80; A61B 17/88; A61C 7/06

(52) U.S. Cl. CPC A61C 11/001; A61B 17/663; A61B 17/8071; A61B 17/8866; A61C 7/06

(57) ABSTRACT

A maxilla holder includes a base plate and a connecting member. One end of the connecting member is connected with the base plate which extends along a pre-designed occlusal plane while the other end of the connecting member includes at least two supporting portions. With the supporting portions abutting against the cranium, the base plate is positioned so that the maxilla is located in a pre-planned position once it is moved onto the base plate.

### CROSS REFERENCE TO RELATED APPLICATIONS

This application is a Divisional of co-pending application Ser. No. 18/813,861, filed on Aug. 23, 2024, for which priority is claimed under 35 U.S.C. § 120; and this application claims priority of application No. 112132179 filed in TAIWAN on Aug. 25, 2023 under 35 U.S.C. § 119 (e), the entire contents of all of which are hereby incorporated by reference.

### BACKGROUND OF INVENTION

### Field of Invention

The present invention relates to a surgical aid tool, particularly to a maxilla holder.

### Related Prior Art

Certain congenital and acquired factors can impact the growth of the maxilla or mandible, leading to conditions such as protrusion of the upper jaw (e.g., overbite) or protrusion of the lower jaw (e.g., underbite). These conditions can result in malocclusion, incomplete chewing of food affecting digestion, and physiological issues. Moreover, they may cause psychological concerns related to facial aesthetics and self-confidence. Corrective jaw surgery is a viable solution to address these issues by adjusting the maxilla and mandible to more suitable positions.

One step in orthognathic surgery involves cutting the maxilla from the cranium and repositioning it appropriately. The maxilla, once cut, becomes free and requires a positioning aid tool to facilitate proper placement. In conventional orthognathic surgery, the mandible 82 is often used to position the maxilla 81 (referenced in FIG. 9). Therefore, the positioning aid tool, an occlusal splint for upper and lower teeth, is designed based on computed tomography scans of the patient's head and entire oral cavity (referenced in FIG. 8). The occlusal splint 7 has lower tooth pits 71 on its bottom surface engaging with the lower teeth of the mandible and upper tooth pits 72 on its top surface located at the anticipated position for the repositioned maxilla. The upper teeth engage with the upper tooth pits 72, providing the necessary positioning (refer to FIG. 9). The method of use for the occlusal splint 7 is as follows:

First, place the occlusal splint 7 on the lower teeth of the mandible, and make sure that the lower teeth occlude with the lower teeth tooth pits 71. Then move the cut maxilla to the upper tooth pits 72 on the occlusal splint 7, and ensure that the upper teeth occludes with the tooth pits 72, when this point has been reached, it indicates that the maxilla has been moved to the intended position, as shown in FIG. 9. Then, by use wiring and fixtures to bind the upper and lower teeth together, ensuring that the maxilla 81 is securely positioned on the mandible 82.

However, the method of using the occlusal splint 7 on the mandible to determine the positioning of the cut maxilla has the following drawbacks:
1. The mandible 82, serving as the positioning base, is not fixed and can move vertically and even forward and backward due to its joint connection with the cranium 83. This lack of stability in the mandible 82 prevents precise positioning, leading to the inability to relocate the maxilla 81 to the intended position accurately.
2. The width W of the occlusal splint 7 is too wide for the small oral space, which makes the already small oral space to become even smaller, this will hinder the entire surgical procedure.
3. Since the mandible 82 is not fixed and is able to move to a limited extent, and because the width W of the occlusal splint 7 is too wide, which makes it challenging to perform the tooth binding procedure. Furthermore, during the binding procedure, the position of the mandible 82 may move due to the binding, which will in turn alter the position of the maxilla 81, preventing it from staying in the correct location originally positioned.

### SUMMARY OF THE INVENTION

To address the aforementioned issues, the present invention provides a maxilla holder that comprises of a base plate extending along a predetermined occlusal plane; a connecting member, one end of which is connected to the base plate; and at least two first supporting portions located at the end of the connecting member, used to support against the cranium.

Preferably, the connecting member includes a stem portion connected to the base plate and at least two branch portions extending in different directions from the stem portion. The two first supporting portions are formed at the end of the branch portions to support against the sides of the piriform aperture.

Furthermore, the two branch portions, apart from the ends, each has a second supporting portion to support against the sides of the anterior nasal spine.

Preferably, a depression facing the cranium is formed at the intersection of the stem portion and the at least two branch portions to accommodate the anterior nasal spine.

Preferably, the base plate forms a U-shape, with the stem portion connected at the central position of the outer edge of the base plate, and the opening angle of the two branch portions does not exceed the opening angle of the base plate.

Preferably, each first supporting portion and each second supporting portion respectively has a perforation.

Preferably, tooth pits are provided on the top surface and bottom surface of the base plate.

### BRIEF DESCRIPTION OF DRAWINGS

FIGS. 1 and 2 are perspective views of the present invention;

FIG. 3 is a front view of the present invention;

FIG. 4 is a side view of the present invention;

FIG. 5 is a perspective view of the present invention from another angle;

FIGS. 6 and 7 illustrate the present invention in an operational state;

FIG. 8 is a plan view of a conventional occlusal splint;

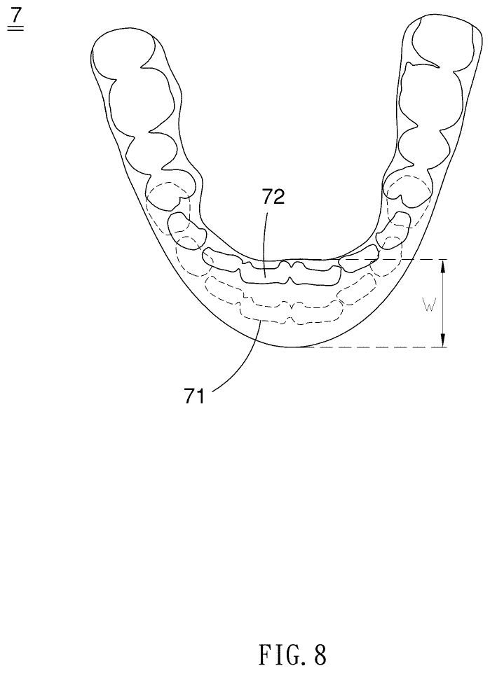

FIG. 9 illustrates a conventional occlusal splint in an operational state.

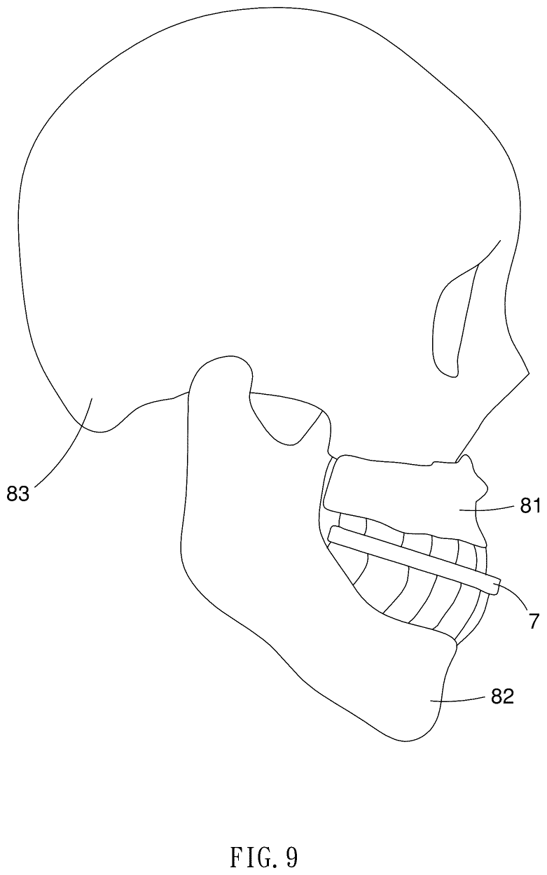

### DETAILED DESCRIPTION OF PREFERRED EMBODIMENT

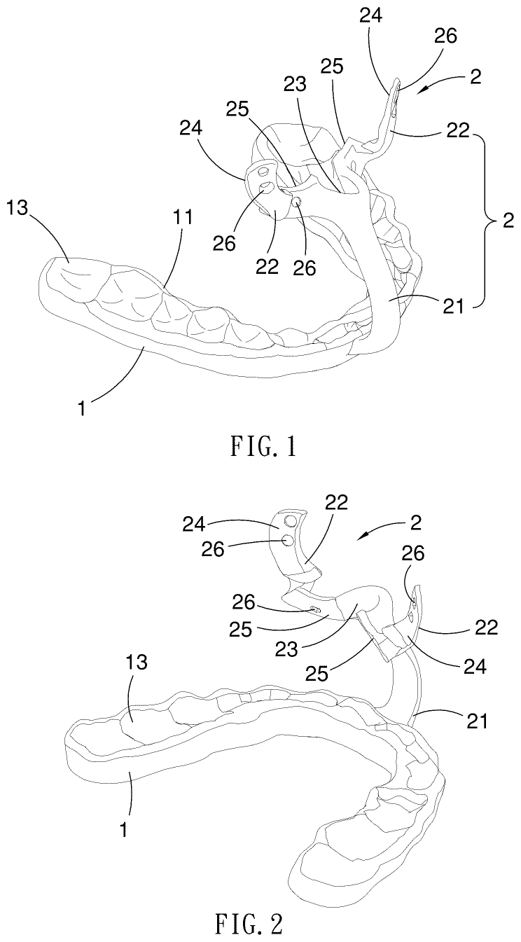

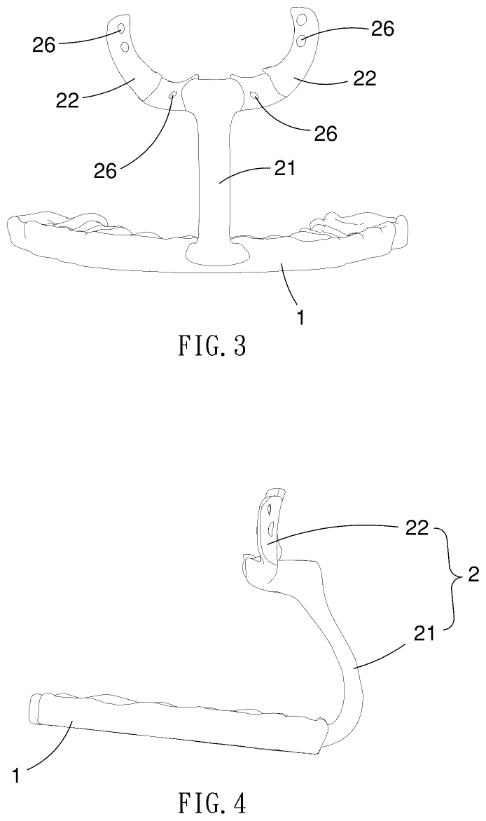

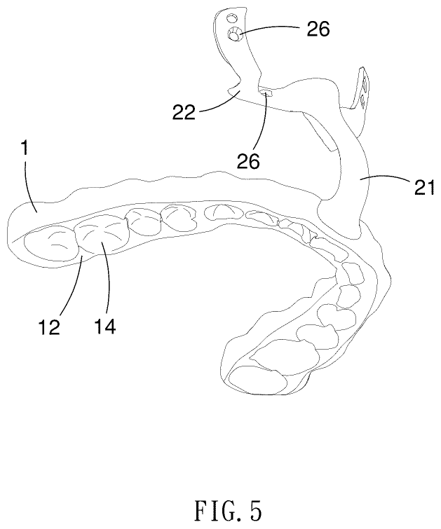

Refer to FIGS. 1 through 5, illustrating the maxilla holder provided by the present invention, comprising a base plate 1 and a connecting member 2. The base plate 1 forms a U-shape corresponding to the arrangement of upper and lower teeth, with a width slightly wider than the width of the teeth. The top surface 11 of the base plate 1 features tooth pits 13 complementary to the upper teeth of the maxilla, allowing the maxilla to be positioned by engaging with the upper teeth into the tooth pits 13. The connecting member 2 includes a stem portion 21 and two branch portions 22. The stem portion 21 is connected at one end to the central position of the outer edge of the base plate 1 and extends upward, while the two branch portions 22 extend in different directions from the top end of the stem portion 21. In this embodiment, the opening angle of the two branch portions 22 does not exceed that of the sides of the base plate 1.

The two branch portions 22 are connected to the stem portion 21, forming a depression 23 at the junction due to bending. The depression 23 faces the rear of the base plate 1 and creates a receiving space for the anterior nasal spine to extend into. After bending to form the depression 23, the two branch portions 22 extend upward and sequentially form a second supporting portion 25 and a first supporting portion 24 along the extension direction, wherein the first supporting portion 24 located at the end of the branch portion 22. The first supporting portion 24 is designed to abut against the sides of the piriform aperture of the cranium, with its surface shape complementary to the shape of the abutting position to ensure proper contact. The second supporting portion 25 is designed to abut against the sides of the anterior nasal spine of the maxilla, with its surface shape complementary to the shape of the abutting position to ensure proper contact.

The specific manufacturing process of the maxilla holder of the present invention begins with a computed tomography scan of the user's skull and entire mouth to construct three-dimensional models of the cranium and upper and lower jaws. Based on these models, the planned position for the detached maxilla to be repositioned is determined. This information, including the relative positions or distances between the planned positions and the cranium, is then used to locate the positions of the base plate 1, first supporting portion 24, and second supporting portion 25 of the present invention. The base plate 1 extends along the planned occlusal plane, the first supporting portion 24 abuts against both sides of the piriform aperture, and the second supporting portion 25 abuts against both sides of the anterior nasal spine. These components are then connected using the connecting member 2. Therefore, the maxilla holder of the present invention is a customized structure, and maxilla holders used by different users do not have identical shapes and sizes. However, each functional component (i.e., the base plate 1, first supporting portion 24, and second supporting portion 25) functions in the same manner to achieve the desired effect.

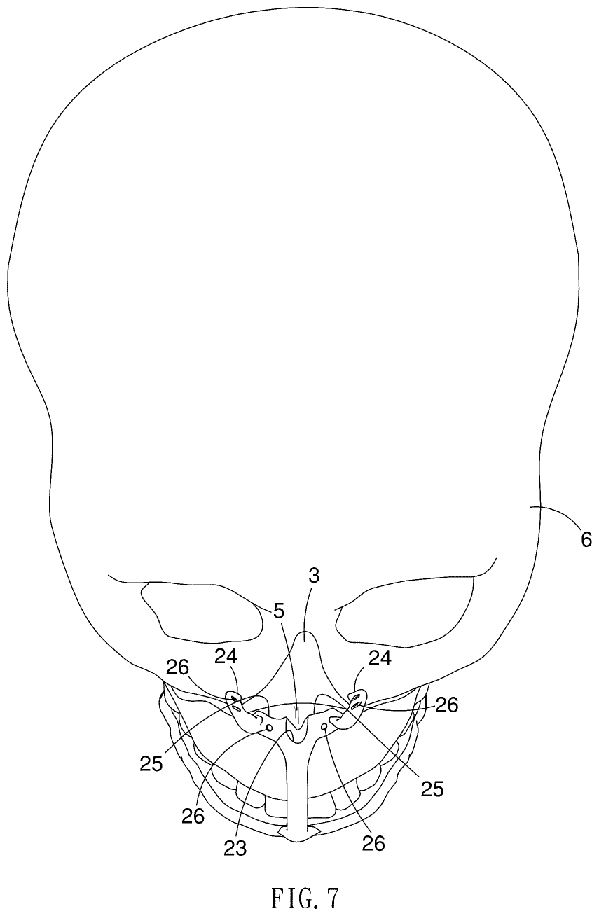

Therefore, during the actual process of positioning the maxilla, due to the fact that the locator of the present invention is designed in advance for the new position of the maxilla, once the locator is inserted into the oral cavity, as shown in FIGS. 6 and 7, simply ensuring that the two first supporting portions 24 respectively abut on both sides of the piriform aperture 3 and achieve stable abutment based on complementary shapes, the base plate 1 will automatically come to the designed position of the maxilla 4. At this point, placing the maxilla 4 onto the base plate 1 and ensuring that both sides of the anterior nasal spine 5 abut against the two second supporting portions 25, where the anterior nasal spine 5 can be accommodated within the depression 23, results in the maxilla 4 reaching the designated position. Subsequently, by engaging the upper teeth into the tooth pits 13 on the top surface 11 of the base plate 1, the maxilla 4 can be easily moved to the desired position and securely positioned.

The present invention achieves stable positioning by having the two first supporting portions 24 abut against the cranium 6. However, if there are more first supporting portions 24 abutting against the cranium 6, the precision of the positioning can be further enhanced. Accordingly, the connecting member 2 of the present invention may also include a greater number of branch portions 22 to form a greater number of first supporting portions 24.

For accurate positioning of the maxilla, the maxilla holder of the present invention may be constructed using metal materials (such as stainless steel) to increase rigidity, ensuring precise positioning without deformation during the procedure. Another advantage of using metal instruments is the ability to reduce thickness while maintaining rigidity, providing the surgeon with ample space for operation during the procedure. Alternatively, the maxilla holder of the present invention may also be made of plastic materials, although the structural thickness may be slightly larger due to rigidity requirements.

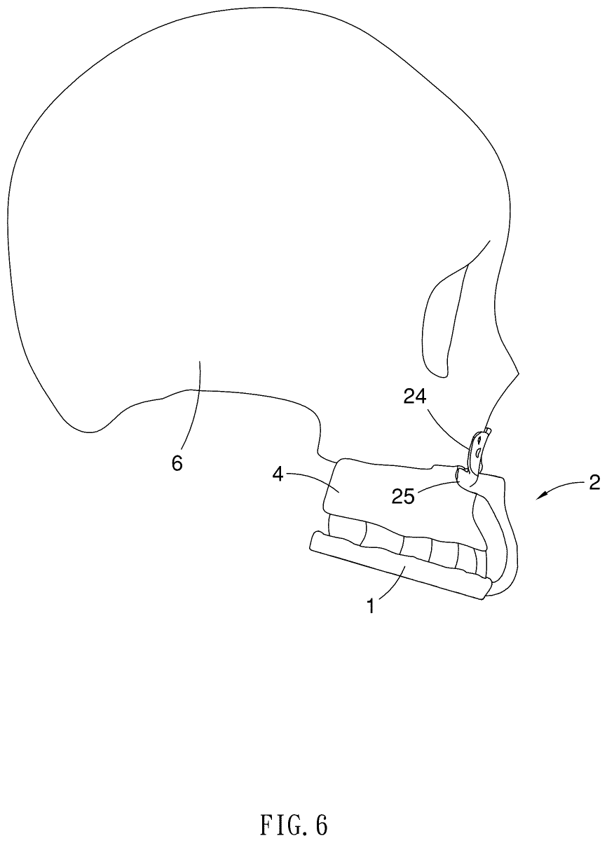

Additionally, as shown in FIG. 5, tooth pits 14 complementary to the shape of the lower teeth may also be provided on the bottom surface 12 of the base plate 1, allowing the base plate 1 to abut against the lower teeth for auxiliary positioning.

Furthermore, perforations 26 can be provided on both the two first supporting portions 24 and the two second supporting portions 25. After the first supporting portions 24 abut against the sides of the piriform aperture 3, and the second supporting portions 25 abut against the sides of the anterior nasal spine 5, bone screws can be inserted through the perforations 26 and into the cranium 6 and maxilla 4. This secures the positioning device of the present invention to the cranium 6 and maxilla 4, thereby strengthening the positioning foundation of the present invention.

In view of the above, the distinguishing feature of the present invention lies in utilizing the fixed cranium 6 as the positioning foundation. When the first supporting portions 24 are abutted against and secured to the cranium 6, they effectively serve as an extension of the cranium 6, allowing the maxilla 4 to be precisely positioned by easily placing it on the base plate 1. This is different from previous techniques that used the movable mandible as the positioning foundation for positioning the maxilla, resulting in more precise positioning.

According to the aforementioned description of the maxilla holder of the present invention, the installation process of the maxilla holder includes the following: Place the two first supporting portions 24 against the two sides of the patient's piriform aperture 3 on the cranium 6. Then insert at least two screws through the perforations 26 of the two first supporting portions 24 and screw them into the cranium 6, so that the two first supporting portions 24 are respectively pressed against and secured on either side of the piriform aperture 3. At this point, the base plate 1 will be positioned within the patient's oral cavity and in the desired correct position. This position, and the relative positions of the base plate 1 and the two first supporting portions 24 are designed based on the results of a computed tomography (CT) scan prior to the making of the maxilla holder. Next, place the cut maxilla 4 onto the base plate 1, as shown in FIG. 6. At this point, the maxilla 4 is positioned in the correct location. Preferably, two screws can also be inserted through the perforations 26 of the two second supporting portions 25 and screwed into the maxilla 4, so that the maxilla 4 is pressed against and fixed to the two second supporting portions 25.

Since the maxilla holder is fixed to the cranium 6 by the two first supporting portions 24 and will not move further, and because the width of the base plate 1 is smaller (as its bottom surface 12 can optionally omit the tooth pits 14), the issues previously arising from using the mandible to position the maxilla can be resolved.

What is claimed is:

1. A method for positioning a maxilla, comprising:
fabricating a holder based on a computed tomography scan content, wherein the computed tomography scan content includes a cranium and a maxilla of a user, and the holder comprises a base plate, a stem portion extending upward from the base plate, a branch portion extending leftward from a top end of the stem portion, and another branch portion extending rightward from the top end of the stem portion, wherein a top surface of the base plate has tooth pits for fitting upper teeth of the maxilla, the top end of the stem portion has a depression for accommodating an anterior nasal spine of the cranium, ends of the two branch portions are respectively provided with a first supporting portion for abutting against two sides of a piriform aperture of the cranium;
accommodating the anterior nasal spine of the cranium in the depression of the holder, and causing the two first supporting portions of the holder to respectively abut against both sides of the piriform aperture of the cranium;
fixing the two first supporting portions of the holder to both sides of the piriform aperture of the cranium; and
placing the detached maxilla onto the base plate of the holder, and fitting the upper teeth of the maxilla into the tooth pits on the base plate.

2. The method for positioning a maxilla as recited in claim 1, wherein each branch portion of the holder has a second supporting portion between the top end of the stem portion and the first supporting portion, and the method includes:
abutting the two second supporting portions of the holder against both sides of the anterior nasal spine of the maxilla.

3. The method for positioning a maxilla as recited in claim 1, wherein the base plate is U-shaped, and the stem portion is connected at the central position of the outer edge of the base plate.

4. The method for positioning a maxilla as recited in claim 1, wherein each of the first supporting portions and each of the second supporting portions has a perforation.

5. The method for positioning a maxilla as recited in claim 1, wherein the computed tomography scan content includes a mandible of the user, a bottom surface of the base plate has tooth pits for fitting the lower teeth of the mandible, and the method includes:
fitting the lower teeth of the mandible into the tooth pits on the bottom surface of the base plate.

6. A method for positioning a maxilla, comprising:
fabricating a holder based on a computed tomography scan content, wherein the computed tomography scan content includes a cranium and a maxilla of a user, and the holder comprises a connecting member and a base plate connected to a bottom end of the connecting member, when the holder is fixed to the cranium and a top end of the connecting member is aligned with an anterior nasal spine of the cranium, the base plate is positioned at a correct location suitable for placing the detached maxilla;
fixing the holder to the cranium such that the top end of the connecting member faces the anterior nasal spine of the cranium;
and placing the detached maxilla onto a top surface of the base plate of the holder.

7. The method for positioning a maxilla as recited in claim 6, wherein the top surface of the base plate has tooth pits for fitting upper teeth of the maxilla, and the step of placing the detached maxilla onto the base plate comprises fitting upper teeth of the maxilla into tooth pits.

8. The method for positioning a maxilla as recited in claim 7, wherein the computed tomography scan content includes a mandible of the user, a bottom surface of the base plate has tooth pits for fitting the lower teeth of the mandible, and the method includes:
fitting the lower teeth of the mandible into the tooth pits on the bottom surface of the base plate.
